SEQUENCE LISTING (32 sequences)

SEQ ID NO: 1 - DNA, 51 bp, Artificial Sequence
  Synthetic
cctgccgttcgacgatgcgatcgcatcatcaataatataccttatagatgg

SEQ ID NO: 2 - DNA, 37 bp, Artificial Sequence
  Synthetic
gatccacatacgaattccggtaatcgaaacctccacg

SEQ ID NO: 3 - DNA, 39 bp, Artificial Sequence
  Synthetic
gaattcgtatgtggatcctgggaaccaccagtaatgtca

SEQ ID NO: 4 - DNA, 53 bp, Artificial Sequence
  Synthetic
cgcggatcttccagagatgcgatcgcatcatcaataatataccttatagatgg

SEQ ID NO: 5 - DNA, 41 bp, Artificial Sequence
  Synthetic
gatatctagagtgaattcgtccaaatgactaatgcaggtgc

SEQ ID NO: 6 - DNA, 49 bp, Artificial Sequence
  Synthetic
ccagtagaagcgccggatttaaataggaaaagtttcgttcttctggttg

SEQ ID NO: 7 - DNA, 48 bp, Artificial Sequence
  Synthetic
attattgactagagtaatttaaatggactaagagacctgctacccatg

SEQ ID NO: 8 - DNA, 41 bp, Artificial Sequence
  Synthetic
gaattcactctagatatcccacttttagctgtagagacccg

SEQ ID NO: 9 - DNA, 44 bp, Artificial Sequence
  Synthetic
tgattattgactagagtatacgtccaaatgactaatgcaggtgc

SEQ ID NO: 10 - DNA, 45 bp, Artificial Sequence
  Synthetic
gatatcatttaaatactagtaggaaaagtttcgttcttctggttg

SEQ ID NO: 11 - DNA, 44 bp, Artificial Sequence
  Synthetic
actagtatttaaatgatatcggactaagagacctgctacccatg

SEQ ID NO: 12 - DNA, 46 bp, Artificial Sequence
  Synthetic
accgcccagtagaagcgccggtgccacttttagctgtagagacccg

SEQ ID NO: 13 - DNA, 39 bp, Artificial Sequence
  Synthetic
gatatctagagtgaattcggggtggagtgtttttgcaag

SEQ ID NO: 14 - DNA, 51 bp, Artificial Sequence
  Synthetic
cgcccagtagaagcgccgggtttaaacgtaatcgaaacctccacgtaatgg

SEQ ID NO: 15 - DNA, 50 bp, Artificial Sequence
  Synthetic
ccagatatacgcgtgtatagtttaaacgagaccggatcatttggttattg

SEQ ID NO: 16 - DNA, 40 bp, Artificial Sequence
  Synthetic
gaattcactctagatatcgggaaatgcaaatctgtgaggg

SEQ ID NO: 17 - DNA, 44 bp, Artificial Sequence
  Synthetic
accgcccagtagaagcgccggtgggggtggagtgtttttgcaag

SEQ ID NO: 18 - DNA, 44 bp, Artificial Sequence
  Synthetic
actagtgtttaaacgatatcgtaatcgaaacctccacgtaatgg

SEQ ID NO: 19 - DNA, 43 bp, Artificial Sequence
  Synthetic
gatatcgtttaaacactagtgagaccggatcatttggttattg

SEQ ID NO: 20 - DNA, 42 bp, Artificial Sequence
  Synthetic
ccagatatacgcgtgtatacgggaaatgcaaatctgtgaggg

SEQ ID NO: 21 - DNA, 45 bp, Artificial Sequence
  Synthetic
agagtgcaccatatggaattcggactaagagacctgctacccatg

SEQ ID NO: 22 - DNA, 23 bp, Artificial Sequence
  Synthetic
gctgtgtggtaattggctgtggg

SEQ ID NO: 23 - DNA, 36 bp, Artificial Sequence
  Synthetic
cattcaaaactaacaatgcagaaacccgcagacatg

SEQ ID NO: 24 - DNA, 40 bp, Artificial Sequence
  Synthetic
gagtctggatcacggctacatgggggtagagtcataatcg

SEQ ID NO: 25 - DNA, 20 bp, Artificial Sequence
  Synthetic
ccgtgatccagactccggag

SEQ ID NO: 26 - DNA, 25 bp, Artificial Sequence
  Synthetic
tgttagttttgaatgagtctgcaaa

SEQ ID NO: 27 - DNA, 44 bp, Artificial Sequence; identical to SEQ ID NO: 17
  Synthetic

SEQ ID NO: 28 - DNA, 44 bp, Artificial Sequence; identical to SEQ ID NO: 18
  Synthetic

SEQ ID NO: 29 - DNA, 43 bp, Artificial Sequence; identical to SEQ ID NO: 19
  Synthetic

SEQ ID NO: 30 - DNA, 42 bp, Artificial Sequence; identical to SEQ ID NO: 20
  Synthetic

SEQ ID NO: 31 - DNA, 32 bp, Artificial Sequence
  Synthetic
agatatacgcgttgacattgattattgactag

SEQ ID NO: 32 - DNA, 23 bp, Artificial Sequence
  Synthetic
gctggttctttccgcctcagaag

(19) United States
(12) Patent Application Publication
(10) Pub. No.: US 2019/0388536 A1
(43) Pub. Date: Dec. 26, 2019

(54) Human Type 14 Replication Defective Adenovirus Vector and Preparation Method for Same and Applications Thereof

(71) Applicant: GUANGZHOU N BIOMED LTD., Guangzhou (CN)

(72) Inventors: Ling Chen, Guangzhou (CN); Liqiang Feng, Guangzhou (CN); Chufang Li, Guangzhou (CN); Xikui Sun, Guangzhou (CN)

(21) Appl. No.: 16/558,301

(22) Filed: Sep. 2, 2019

Related U.S. Application Data
(63) Continuation of application No. PCT/CN2017/082901, filed on May 3, 2017.

(30) Foreign Application Priority Data
Mar. 1, 2017 (CN) 201710116966.5

(51) Int. Cl. A61K 39/235; C12N 15/66; C12N 15/86; A61P 31/20

(52) U.S. Cl. CPC A61K 39/235; A61P 31/20; C12N 15/86; C12N 15/66

(57) ABSTRACT

The present invention provides a human type 14 replication defective adenovirus vector, and a preparation method for the same, the method comprising: constructing an Ad14 genome into a plasmid, with knocking out E3 and E1 genes of the Ad14 genome, and replacing open reading frames 2, 3, 4, 6, and 6/7 of an E4 gene of the Ad14 genome with corresponding reading frames of an Ad5 genome. The human type 14 replication defective adenovirus vector according to the present invention is potentially applicable in the research and development of a vaccine and a drug against human type 14 adenovirus infection, applicable as a gene vector in the research and development of other pathogen vaccines, and applicable in a biological report and trace system, etc.

### CROSS-REFERENCE TO RELATED APPLICATIONS

The present application is a Continuation Application of PCT application No. PCT/CN2017/082901 filed on May 3, 2017, which claims the benefit of Chinese Patent Application No. 201710116966.5 filed on Mar. 1, 2017, the contents of which are hereby incorporated by reference.

### REFERENCE TO SEQUENCE LISTING

The Sequence Listing is submitted as an ASCII formatted text filed via EFS-Web, with a file name of “Sequence_Listing.TXT”, a creation date of Sep. 2, 2019, and a size of 6,119 bytes. The sequence listing filed via EFS-Web is part of the specification and is incorporated in its entirety by reference herein.

### FIELD OF THE INVENTION

The present invention relates to biotechnical field, and particularly relates to a human type 14 replication defective adenovirus vector and a preparation method for the same and applications thereof.

### BACKGROUND OF THE INVENTION

Adenovirus (Ad) is a double stranded DNA virus with a genome length of 34 to 38 kb. Recently reported adenovirus includes 69 genotypes, which can be classified into seven subgroups from A to G Adenovirus not only causes asymptomatic infections, but also induces respiratory diseases, eye diseases, gastroenteritis, etc. Type 3 and type 7 adenoviruses in the subgroup B and type 4 adenovirus in the subgroup E usually induce serious respiratory diseases. For over half a century since 1955 when the type 14 adenovirus was first discovered in Dutch troops, the type 14 adenovirus did not cause serious respiratory diseases or wide prevalence. However, since 2006 when the type 14 adenovirus had been reported by the U.S. troops, the type 14 adenovirus frequently caused infections and death in the United States. Thereafter, the type 14 adenovirus caused infections and death in both Ireland in Europe and Canada in the North America. In 2011, infections caused by the type 14 adenovirus also broke out in a secondary school in Gansu province, China. In view of the prevalence and high pathogenicity of the type 14 adenovirus, there are currently no vaccines and drugs for prevention and treatment of infections caused by the type 14 adenovirus. Therefore, the research and development of vaccines and drugs against the type 14 adenovirus are of great significance to human health.

Since 1970 s, the U.S. troops started to useenteric-coated live vaccines against type 4 and type 7 adenoviruses, which have provided good protection. As vaccines against the type 4 and type 7 adenoviruses have achieved good preventive effects, adenovirus vaccines were stopped to be produced in 1996, and adenovirus vaccines in the inventory were exhausted in 1999. Then, respiratory diseases caused by adenovirus infections broke out again in the U.S. troops, so the vaccines against type 4 and type 7 adenovirus had to be reproduced. Since the available adenovirus vaccine is a live vaccine against type 4 and type 7 adenoviruses used in the U.S. troops, there is no exact preventive effect on the recent outbreak of a type 14 adenovirus. On the other hand, this vaccine is mainly comprised of a wild-type adenovirus, and residual live viruses discharged from the intestinal tract very easily pollute water resources and thus result in the spread of the viruses; due to the potential safety risk of this vaccine, it cannot be applied to common population. Safe and effective vaccines against type 14 replication defective adenovirus are urgently needed.

Replication defective adenovirus vectors are widely used for research and development of vaccines and gene therapy, which are not only highly safe, but also capable of inducing strong immunoreaction in vivo. An E1 gene of the adenovirus is essential for the replication and propagation of the adenovirus, whereas an E3 gene of the adenovirus antagonizes host's immune system. The adenovirus with the E1 and E3 genes knocked out concurrently loses the capability of replicating in normal individuals and exhibits an attenuated phenotype, but the major surface antigens, such as Hexon and Fibre, of the adenovirus are not affected. A replication defective adenovirus vaccine is not only highly safe, but also induces an immunoreaction against the adenovirus in vivo. The replication defective adenovirus can be propagated in complementary cell lines, such as 293 and PerC6 cells expressing the Ad5 E1 gene. Nonetheless, adenoviruses from subgroup B with the E1 and E3 genes thereof knocked out are difficult to propagate in these cell lines on the main grounds that Ad5 E1B 55K cannot interact with E4 Orf6 adenovirus from subgroup B, and cannot effectively inhibit nucleus export of host cell mRNA and enhance the expression of late viral proteins.

Studies have reported that Ad26 and Ad35, in which E4 ORF6 of Ad26 and Ad35 was replaced by E4 ORF6 of Ad5 while knocking out the E1 gene of Ad26 and Ad35, can still be replicated in 293 and PerC6 cells and propagate effectively. There is still no way to obtain a large amount of replication defective Ad14 at present. The E4 gene contains reading frames, e.g., Orf 1, Orf2, Orf3, Orf4, 34K(Orf6), Orf6/7 and the like, and as the proteins encoded by the E4 gene may all interact with the proteins encoded by the E1 gene, the E4 gene plays a significant role in the process of adenovirus replication and packaging.

Gene therapy carried out by adenovirus vectors has been clinically tested on a large scale. The proportion of clinical trials conducted with adenoviral vectors worldwide has currently reached 22.2%, ranking first among all types of vectors. Adenovirus vectors are mainly used in Ad2 and Ad5. Since humans are naturally infected with adenovirus, most of people have antibodies against Ad2 and Ad5, which limits the use of conventional adenovirus vectors. Neutralizing antibodies of Ad2 and Ad5 are of high seropositive rate, even up to 90%, in developing countries and regions such as Africa, South America and China. The presence of the neutralizing antibodies inhibits the entry of adenovirus into somatic cells and disables immunological or therapeutic functions of the adenovirus. In order to evade the immunoreaction of in vivo pre-existing antibodies against adenovirus, researchers have tried a series of strategies, including: 1) modifying or reconstructing the capsid protein on the surface of adenovirus to let adenovirus escape from the pre-existing immunoreaction; 2) inhibiting immunoreaction against adenovirus by using an immunity inhibitor such as cyclosporine, cyclophosphamide, and FK506; 3) developing rare adenovirus vectors, such as chimpanzee adenoviruses CV-68 and 63, without response to a pre-existing antibody; and 4) in vitro infecting PBMC by adenovirus, and then performing autologous transfusion (an AVIP technique we previously developed), and so on. Ad14 has not been widely prevailed in human population and as a neutralizing antibody against Ad14 is lacking in the human body, a human type 14 adenovirus vector is a good substitute for vectors such as Ad2 and Ad5.

### SUMMARY OF THE INVENTION

The technical problems to be solved by the present invention is to provide a human type 14 replication defective adenovirus vector to overcome the above problems existing in the prior art.

The above technical problems will be resolved by the following technical solutions:

A human type 14 replication defective adenovirus vector, prepared by the following method: constructing an Ad14 genome into a plasmid, with knocking out E1 and E3 genes of the Ad14 genome, and replacing open reading frames 2, 3, 4, 6, and 6/7 of an E4 gene of the Ad14 genome with corresponding reading frames of an Ad5 genome.

Preferably, the human type 14 replication defective adenovirus vector further integrates an exogenous gene expression cassette into an E1 gene region of Ad14.

A method of preparing the human type 14 replication defective adenovirus vector, comprising the following steps:

S1. obtaining left and right ends of the Ad14 genome by PCR amplification, ligating the ends into an ampicillin resistant plasmid to obtain pT-Ad14(L+R), linearizing the pT-Ad14(L+R), and recombining the linearized pT-Ad14(L+R) with the Ad14 genome to obtain a genomic plasmid pAd14;

S2. obtaining left and right arms of an Ad14 E3 gene by PCR amplification, ligating the arms in a reverse direction into a kanamycin resistant plasmid, linearizing the plasmid, and obtaining a genomic plasmid pAd14ΔE3-Kana with the E3 gene of Ad14 knocked out, through homologous recombination of the linearized plasmid with pAd14 which is linearized by partial enzyme digestion;

S3. obtaining left and right arms of an Ad14 E3 gene by PCR amplification, ligating the arms in a forward direction into the kanamycin resistant plasmid, linearizing the plasmid, and obtaining a genomic plasmid pAd14ΔE3 with a kanamycin resistant gene knocked out, through recombination of the linearized plasmid with the linearized pAd14ΔE3-Kana;

S4. obtaining left and right arms of an Ad14 E1 gene by PCR amplification, ligating the arms in a reverse direction into a kanamycin resistant plasmid, linearizing the plasmid, and obtaining a genomic plasmid pAd14ΔE1ΔE3-Kana with the E1 gene of Ad14 knocked out, through homologous recombination of the linearized plasmid with pAd14ΔE3 which is linearized by partial enzyme digestion;

S5. obtaining left and right arms of an Ad14 E1 gene by PCR amplification, ligating the arms in a forward direction into a kanamycin resistant plasmid, linearizing the plasmid, and obtaining a genomic plasmid pAd14ΔE1ΔE3 with a kanamycin resistant gene knocked out, through recombination of the linearized plasmid with the linearized pAd14ΔE1ΔE3-Kana; and

S6. obtaining an Ad14 E4 gene by PCR amplification and ligating the Ad14 E4 gene into an ampicillin resistant plasmid to obtain p14E4; obtaining open reading frames 2 to 6/7 of E4 gene of an Ad5 genome by PCR amplification, replacing corresponding regions of the Ad14 E4 gene to obtain p14E4(5E4), linearizing the p14E4(5E4), and obtaining a genomic plasmid pAd14ΔE1ΔE3(5E4) with E1 and E3 genes knocked out and E4 gene replaced, through homologous recombination of the linearized p14E4(5E4) with the linearized pAd14ΔE1ΔE3.

Preferably, the step Si comprises:

obtaining left and right ends L-Ad14 and R-Ad14 as recombination arms of the Ad14 genome by PCR amplification using the Ad14 genome as a template, ligating the arms into a linearized T vector to obtain pT-Ad14(L+R), while introducing EcoRI and BamHI sites as enzyme digestion sites between left and right arms of the pT-Ad14(L+R), digesting the pT-Ad14(L+R) with EcoRI+BamHI through double enzyme digestion, and recombining with the Ad14 genome after linearization of the double enzyme digestion to obtain pAd14.

Most preferably, in one of the examples, the step Si comprises:

obtaining left and right ends L-Ad14 and R-Ad14 as recombination arms of the Ad14 genome by PCR amplification using the Ad14 genome as a template, ligating the arms into a linearized T vector to obtain pT-Ad14(L+R), while introducing EcoRI and BamHI sites as enzyme digestion sites between left and right arms of the pT-Ad14(L+R), digesting the pT-Ad14(L+R) with EcoRI+BamHI through double enzyme digestion, and recombining with the Ad14 genome after linearization of the double enzyme digestion to obtain pAd14.

Preferably, the step S2 comprises:

obtaining homologous recombination arms L-ΔE3 and R-ΔE3 of the E3 gene by PCR amplification using the Ad14 genome as a template, ligating the arms in a reverse direction into a pVax vector to obtain pVax-ΔE3(L+R), linearizing the pVax-ΔE3(L+R), and obtaining a plasmid pAd14ΔE3-Kana with the E3 gene knocked out and a unique linearized enzyme digestion site SwaI introduced in the E3 gene region, through homologous recombination of the linearized pVax-ΔE3(L+R) with pAd14 linearized by partial enzyme digestion using EcoRI and a dual-resistance screening by ampicillin and kanamycin.

Most preferably, in one of the examples, the step S2 comprises:

obtaining left and right arms L-ΔE3 and R-ΔE3 of the E3 gene by PCR amplification using the Ad14 genome as a template, and obtaining a pVax skeleton by PCR amplification using a pVax vector as a template, conducting a triple-fragment ligation of the pVax skeleton with an Exnase recombinase to obtain pVax-ΔE3 (L+R); digesting the pVax-ΔE3 (L+R) with EcoRI+EcoRV through double enzyme digestion and digesting the pAd14 with EcoRI through restriction enzyme digestion, and obtaining pAd14ΔE3-Kana through homologous recombination of fragments resulting from recovery of both enzyme digested products.

Preferably, the step S3 comprises:

obtaining homologous recombination arms L-ΔK(E3) and R-ΔK(E3) of the E3 gene by PCR amplification using the Ad14 genome as a template, ligating the arms in a forward direction into a pVax vector to obtain pVax-ΔK(E3), linearizing the pVax-ΔK(E3), and obtaining pAd14ΔE3 with E3 and kanamycin resistant genes knocked out and a single enzyme digestion site SwaI introduced, through recombination of the linearized pVax-ΔK(E3) with pAd14ΔE3-Kana linearized by SwaI.

Most preferably, in one of the examples, the step S3 comprises:

obtaining left and right arms L-ΔK(E3) and R-ΔK(E3) of the E1 gene by PCR amplification using the Ad14 genome as a template, and obtaining a pVax skeleton by PCR amplification using a pVax vector as a template, conducting a triple-fragment ligation of the pVax skeleton with an Exnase recombinase to obtain pVax-ΔK(E3); digesting the pVax-ΔK(E3) with Bstz17I +SgrAI through double enzyme digestion and digesting pAd14ΔE3-Kana with SwaI through enzyme digestion, linearizing the enzyme digested products, and obtaining pAd14ΔE3 through homologous recombination of fragments resulting from recovery of both enzyme digested products.

Preferably, the step S4 comprises:

according to the same principle as in step S2, obtaining homologous recombination arms L-ΔE1 and R-ΔE1 of the E1 gene by PCR amplification, ligating in reverse direction into a pVax vector to obtain pVax-ΔE1(L+R), linearizing the pVax-ΔE1(L+R), and obtaining a plasmid pAd14ΔE1ΔE3-Kana with the E1 gene knocked out and a unique linearized enzyme digestion site PmeI introduced in the E1 gene region, through homologous recombination of the linearized pVax-ΔE1(L+R) with pAd14ΔE3 linearized by enzyme digestion using PacI and a dual-resistance screening by ampicillin and kanamycin.

Most preferably, in one of the examples, the step S4 comprises:

obtaining left and right arms L-ΔE1 and R-ΔE1 of the E1 gene by PCR amplification using the Ad14 genome as a template, and obtaining a pVax skeleton by PCR amplification using a pVax vector as a template, conducting a triple-fragment ligation of the pVax skeleton with an Exnase recombinase to obtain pVax-ΔE1(L+R); digesting the pVax-ΔE1(L+R) with EcoRI+EcoRV through double enzyme digestion and digesting pAd14ΔE3 with PacI through restriction enzyme digestion, and obtaining pAd14ΔE1ΔE3-Kana through homologous recombination of fragments resulting from recovery of both enzyme digested products.

Preferably, the step S5 comprises:

according to the same principle as in step S3, obtaining homologous recombination arms L-ΔK(E 1) and R-ΔK(E 1) of the E1 gene by PCR amplification, ligating the arms forward into a pVax vector to obtain pVax-ΔK(E1), linearizing the pVax-ΔK(E1), and obtaining pAd14ΔE1ΔE3 with E1 and kanamycin resistant genes knocked out and a single enzyme digestion site PmeI introduced, through recombination of the linearized pVax-ΔK(E1) with pAd14ΔE1ΔE3-Kana linearized by PmeI.

Most preferably, in one of the examples, the step S5 comprises:

obtaining left and right arms L-ΔK(E1) and R-ΔK(E1) of the E1 gene by PCR amplification using the Ad14 genome as a template, and obtaining a pVax skeleton by PCR amplification using a pVax vector as a template, conducting a triple-fragment ligation of the pVax skeleton with an Exnase recombinase to obtain pVax-ΔK(E1); digesting the pVax-ΔK(E1) with Bstz 17I +SgrAI through double enzyme digestion and digesting pAd14ΔE14E3-Kana with PacI through enzyme digestion, linearizing the enzyme digested products, and obtaining pAd14ΔE14E3 through homologous recombination of fragments resulting from recovery of both enzyme digested products.

Preferably, the step S6 comprises:

obtaining Ad5 E4 Orf2-6 and Ad14 E4 by PCR amplification using Ad5 genome and Ad14 genome as templates respectively, ligating the Ad14 E4 into a T vector to obtain p14E4, further knocking out Ad14 E4 Orf2-6 by PCR using p14E4 as a template, then ligating the PCR product with Ad5 E4 Orf2-6 to obtain p14E4(5E4), linearizing the p14E4(5E4), and obtaining pAd14ΔE1ΔE3(5E4), through homologous recombination of the linearized p14E4(5E4) with the linearized pAd14ΔE1E3.

Most preferably, in one of the examples, the step S6 comprises:

Obtaining an Ad5 E4 Orf2-6 gene and an Ad14 E4 gene by PCR amplification using the Ad5 and Ad14 genomes as templates, respectively, and ligating the Ad14 E4 gene into a T vector to obtain p14E4; obtaining a skeleton sequence of p14E4 by PCR amplification using the p14E4 as a template, ligating the p14E4 skeleton sequence into Ad5 E4 Orf2-6 with an Exnase enzyme to obtain p14E4(Ad5Orf2-6); obtaining pAd14ΔE14E3(5E4) through recombination of p14E4(Ad5Orf2-6) linearized with EcoRI and pAd14ΔE14E3 linearized with PsiI.

A method of preparing the human type 14 replication defective adenovirus vector, further comprising the following step:

S7. obtaining homologous recombination arms L-SE1 and R-SE1 of the E1 region by PCR using the Ad14 genome as a template, enzyme digesting the arms and ligating the enzyme digested arms into a pVax vector to obtain pSE1LR; producing an exogenous gene expression cassette CMV-EGFP-BGH by PCR using pGAl-EGFP as a template, enzyme digesting the CMV-EGFP-BGH and pSE1LR, ligating the digested CMV-EGFP-BGH and pSE1LR to obtain pGK141-EGFP, linearizing the pGK141-EGFP, and obtaining Ad14ΔE1ΔE3(5E4) and Ad14ΔE1ΔE3(5E4)-EGFP through homologous recombination of the linearized pGK141-EGFP with the linearized pAd14ΔE1ΔE3(5E4), and then transfecting a cell after further linearization, culturing the transfected cell and obtaining Ad14ΔE1ΔE3(5E4)-EGFP by centrifugal purification.

Preferably, the step S7 comprises:

obtaining a pVax skeleton, upstream and downstream homologous recombination arms L-SE1 and R-SE1 in an E1 region, and an exogenous gene expression cassette CMV-EGFP-BGH by PCR amplification using pVax, the Ad14 genome and pGAl-EGFP as templates respectively, ligating the pVax skeleton, SE1L and SE1R to obtain pSE1LR, and obtaining a target shuttle plasmid pGK141-EGFP harboring an exogenous gene expression cassette through enzyme digestion and ligation of CMV-EGFP-BGH and pSE1LR; [48] linearizing the pGK141-EGFP, and obtaining pAd14ΔE14E3(5E4)-EGFP through homologous recombination of the linearized pGK141-EGFP with the linearized pAd14ΔE14E3(5E4);

enzyme digesting the pAd14ΔE14E3(5E4) and the pAd14ΔE14E3(5E4)-EGFP with AsisI, linearizing the enzyme digestion, transfecting 293 cells for rescue of virus and amplification culture, and obtaining purified Ad14ΔE14E3(5E4) and Ad14ΔE14E3(5E4)-EGFP.

More preferably, in one of the examples, the step S7 comprises:

obtaining a pVax skeleton, upstream and downstream homologous recombination arms L-SE1 and R-SE1 in an E1 region, and an exogenous gene expression cassette CMV-EGFP-BGH by PCR amplification using pVax, the Ad14 genome and pGA1-EGFP as templates respectively, and obtaining pSE1LR through a triple-fragment ligation of the pVax skeleton, L-SE1 and R-SE1 with an Exnase enzyme; enzyme digesting the CMV-EGFP-BGH with SpeI, enzyme digesting the pSE1LR with SpeI+EcoRV, and ligating the enzyme digested CMV-EGFP-BGH and the enzyme digested pSE1LR to obtain a shuttle plasmid pGK141-EGFP;

enzyme digesting the pGK141-EGFP with Bstz17I+SgrAI and linearizing the enzyme digested pGK141-EGFP, linearizing the pAd14ΔE14E3(5E4) with PmeI, linearizing the enzyme digested pAd14ΔE14E3(5E4), and obtaining pAd14ΔE14E3(5E4)-EGFP through homogenous recombination of the linearized pGK141-EGFP and the linearized pAd14ΔE14E3 (5 E4);

enzyme digesting the pAd14ΔE14E3 (5E4) and the pAd14ΔE14E3(5E4)-EGFP with AsisI, linearizing the enzyme digestion, transfecting 293 cells for rescue of virus and amplification culture after recovery by ethanol precipitation, and obtaining pAd14ΔE14E3(5E4) and Ad14ΔE14E3(5E4)-EGFP purified by CsCl₂ density gradient centrifugation.

Use of the human type 14 replication defective adenovirus vector in the preparation of a vaccine.

Use of the human type 14 replication defective adenovirus vector in the preparation of a neutralizing antibody.

Use of the human type 14 replication defective adenovirus vector in the preparation of a biological report and trace system.

Use of the human type 14 replication defective adenovirus vector in the preparation of a vaccine against human type 14 adenovirus.

Use of the human type 14 replication defective adenovirus vector in the preparation of a drug against human type 14 adenovirus.

Advantageous effects: (1) the present invention is succeeded in preparing a human type 14 replication defective adenovirus vector capable of propagating in helper cell lines such as 293 and PerC6, and being purified by density gradient centrifugation, and exhibiting an attenuated phenotype for the lack of replication capacity in normal human cells; (2) the recombinant vector can also express exogenous genes in target cells efficiently; and (3) the recombinant vector of the present invention can be used as a vector for vaccines or gene therapy, and can also be used in research and development of drugs and neutralizing antibodies, as well as in report and trace systems, etc.; the test data in the examples show that the human type 14 replication defective adenovirus vector can induce a higher-level neutralizing antibody against Ad14 at the early stage of immunizing mice.

### DESCRIPTION OF THE DRAWINGS

FIG. 1A shows the principle of Ad14 genome cyclization through homogenous recombination. FIG. 1B is enzyme digestion identification result of pT-Ad14(L+R) with BamH I and Ndell(M1: DNA Ladder 2000; M2: DNA Ladder 15000; 1# to 6#: different pT-Ad14(L+R) clones). FIG. 1C are enzyme digestion identification results of pAd14 with BamH I and pAd14 with EcoR I and Hind III (M2: DNA Ladder 15000; 1# to 6#: different pAd14 clones).

FIG. 2A shows the principle of knocking out the E3 gene of Ad14 through a dual-resistance screening by ampicillin and kanamycin. FIG. 2B is PCR identification result of pVax-ΔE3(L+R) (M1: DNA Ladder 2000; M2: DNA Ladder 15000; 1# to 6#: different pVax-ΔE3(L+R) clones). FIG. 2C is PCR identification result of pAd14ΔE3-Kana (M2: DNA Ladder 15000; 1# to 6#: different pAd14ΔE3-Kana clones).

FIG. 3A shows the principle of knocking out kanamycin resistance from a pAd14ΔE3-Kana plasmid. FIG. 3B is enzyme digestion identification result of pVax-ΔK(E3) with Mlul and EcoRV (M1: DNA Ladder 2000; M2: DNA Ladder 15000; 1# to 4#: different pVax-ΔK(E3) clones). FIG. 3C are results of enzyme digestion of pAd14ΔE3 with HindIII and EcoRV and pAd14ΔE3 with BamH I (M2: DNA Ladder 15000; 1# to 6#: different pAd14ΔE3 clones).

FIG. 4A shows the principle of knocking out the E1 gene of pAd14ΔE3 through a dual-resistance screening by ampicillin and kanamycin. FIG. 4B is PCR identification result of a shuttle plasmid with the E1 gene of pAd14ΔE3 knocked out (pVax-ΔE1(L+R)) (M1: DNA Ladder 2000; M2: DNA Ladder 15000; 1# to 6#: different pVax-ΔE1(L+R) clones). FIG. 4C is enzyme digestion result of pAd14ΔE1ΔE3-Kana with BamH I (M2: DNA Ladder 15000; 1# to 3#: different pAd14ΔE1ΔE3-Kana clones).

FIG. 5A shows the principle knocking out kanamycin resistance from the pAd14ΔE1ΔE3-Kana plasmid. FIG. 5B is result of enzyme digestion of shuttle plasmid with kanamycin resistance knocked out from the E1 region of pAd14ΔE3 (pVax-ΔK(E1)) (M1: DNA Ladder 2000; 1# to 6#: different pVax-ΔK(E1) clones). FIG. 5C is results of enzyme digestion of pAd14ΔE1ΔE3 with BamH I and pAd14ΔE1ΔE3 with HindIII and EcoRV (M2: DNA Ladder 15000; 1# to 6#: different pAd14ΔE1ΔE3 clones).

FIG. 6A shows the principle of replacing ORF2-6 in the E4 gene of pAd14ΔE1ΔE3 with a corresponding sequence of ORF2-6 in the E4 gene of AdS. FIG. 6B is result of enzyme digestion of a shuttle plasmid with ORF2-6 of the Ad14 E4 gene replaced (p14E14 (5Orf2-6))(M2: DNA Ladder 15000; 1# to 6#: different p14E14 (5Orf2-6) clones). FIG. 6C is results of enzyme digestion of pAd14ΔE1ΔE3(5E4) with BamH I and pAd14ΔE1ΔE3(5E4) with HindIII and EcoRV (M2: DNA Ladder 15000; 1# to 6#: different pAd14ΔE1ΔE3(5E4) clones).

FIG. 7A shows the schematic diagram of construction process of pAd14ΔE1ΔE3(5E4)-EGFP harboring an exogenous gene cassette. FIG. 7B is result of enzyme digestion of pGK141-EGFP with HindIII and XbaI (M1: DNA Ladder 2000; M2: DNA Ladder 15000; 1# to 4#: different pGK141-EGFP clones). FIG. 7C is result of enzyme digestion of pAd14ΔE1ΔE3(5E4)-EGFP with HindIII and EcoRV (M2: DNA Ladder 15000; 1# to 6#: different pAd14ΔE1ΔE3(5E4)-EGFP clones).

FIG. 8A shows production result of a type 14 replication defective vector. FIG. 8B shows purification results of a type 14 replication defective vector.

FIG. 9 is results of virus plaques formed by type 14 replication defective vector in 293 and A549 cells.

FIG. 10A shows the time diagram of neutralizing antibody measurement. FIG. 10B is the experimental scheme of immunogenicity assessment of type 14 replication defective vector in vivo. FIG. 10C is detection result of neutralizing antibody titer after immunizing mice for two weeks by replication defective Ad14ΔE1ΔE3(5E4). FIG. 10D is detection result of neutralizing antibody titer after immunizing mice for six weeks by replication defective Ad14ΔE1ΔE3(5E4).

### DETAILED DESCRIPTION OF THE INVENTION

The present invention discloses a method of preparing a human type 14 replication defective adenovirus vector. The preparation method and construction idea of the adenovirus vector in the present invention are applicable to research and development of vaccines against adenoviruses and other pathogenic viruses, screening of drugs and neutralizing antibodies against adenoviruses, and biological report and trace systems.

The term “human type 14 adenovirus” used herein refers to a type 14 adenovirus known to one skilled in the art, and the Ad14 genome used in the examples is also derived from these known human type 14 adenoviruses. The human type 14 replication defective adenovirus vector used herein is not limited to the particular clinical isolates employed in the examples.

The term “exogenous sequence” used herein refers to any DNA sequence not derived from type 14 adenoviruses. It should be understandable to one skilled in the art that the exogenous sequence may be an exogenous gene expression cassette, or a shRNA or miRNA expression cassette, or the like.

In the following examples, the exogenous gene expression cassette may comprise a eukaryotic promoter, an exogenous gene coding sequence and a transcription terminator, as one skilled in the art understood. The exogenous gene coding sequence may be, but is not limited to, coding sequences of green fluorescent proteins, other viral antigens, and shRNA, etc.

To facilitate a clearer understanding of the technical content of the present invention, the following examples are illustrated in conjunction with appended drawings. It is understood that the examples are merely intended to illustrate the present invention, instead of limiting the scope of the present invention. The following examples without experimental methods specified are generally carried out under conventional conditions, such as those described in Molecular Cloning: Laboratory Manual (Sambrook, et al., 1989, New York, Cold Spring Harbor Laboratory Press), or suggested by the manufacturers. Chemical agents used in the examples are commercially available.

Unless otherwise defined, all technical and scientific terminologies used herein have the same meaning as commonly understood by one skilled in the art. The terminologies used in the specification of the present invention are intended to describe particular examples only, instead of limiting the present invention.

### EXAMPLE 1

### Cyclization of Ad14 Genome

1. Construction of a shuttle plasmid pT-Ad14(L+R) for cyclizing the Ad14 genome

A left arm (L-Ad14) and a right arm (R-Ad14) of the Ad14 genome were obtained by PCR amplification using an Ad14 genome as a template.

L-Ad14 primer:

L-Ad14-F,

(SEQ ID NO: 1)

CCTGCCGTTCGACGATGCGATCGCATCATCAATA
ATATACCTTATAGATGG;
L-Ad14-R,

(SEQ ID NO: 2)

GATCCACATACGAATTCCGGTAATCGAAACCTC
CACG.

PCR conditions: 95° C., 3 min; 95° C., 30 s; 57° C., 30 s; 72° C., 30 s; cycles 30; 72° C., 5 min; stored at 12° C.

R-Ad14 primer:

R-Ad14-F,

(SEQ ID NO: 3)

GAATTCGTATGTGGATCCTGGGAACCACCAGTA
ATGTCA;
R-Ad14-R,

(SEQ ID NO: 4)

CGCGGATCTTCCAGAGATGCGATCGCATCATCA
ATAATATACCTTATAGATGG.

PCR conditions: 95° C., 3 min; 95° C., 30 s; 57° C., 30 s; 72° C., 1 min; cycles 30; 72° C., 5 min; stored at 12° C.

2. Construction of pAd14

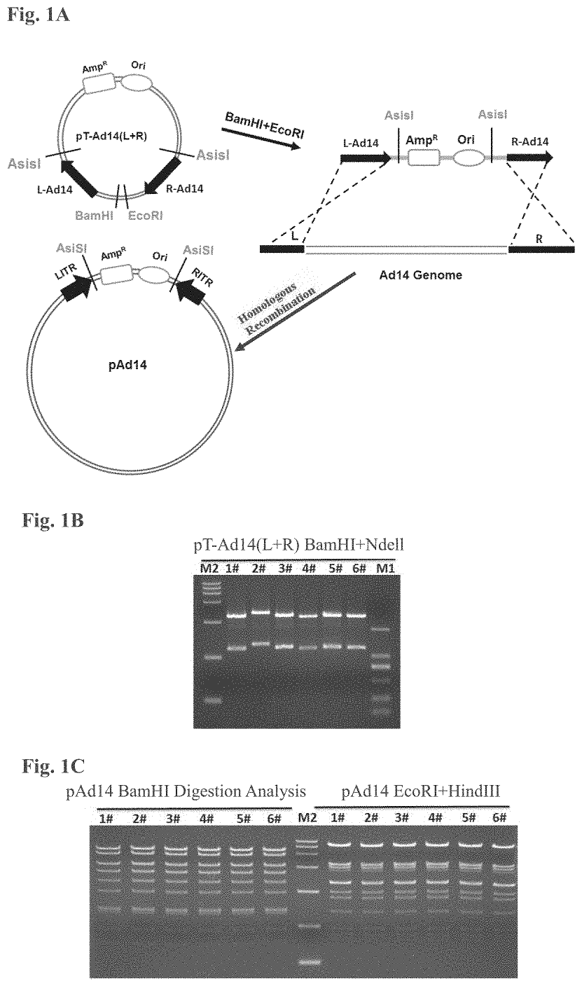

The pT-Ad14(L+R) was linearized by enzyme digestion with EcoRI+BamHI, and then co-transformed into a BJ5183 competent cell together with the Ad14 genome for recombination. The recombined competent cell was subjected to resistance screening by an ampicillin resistant plate, and after amplification of the screened monoclone, a plasmid was extracted from the monoclone and transformed into an XL competent cell, from which a plasmid was extracted to obtain pAd14. The pAd14 was identified by different ways of enzyme digestion. The pAd14 genome had two AsisI enzyme digestion sites introduced at both sides of the pAd14 genome so as to facilitate subsequent linearization and rescue of virus of the reconstructed Ad14 genome. See a schematic of plasmid construction in FIG. 1A, endonuclease digestion result of pT-Ad14(L+R) with EcoRI and BamHI in FIG. 1B, and endonuclease digestion results of pAd14 with BamHI and pAd14 with EcoRI and HindIII in FIG. 1C.

### EXAMPLE 2

Knocking out of E3 gene and construction of pAd14ΔE3-Kana plasmid

1. Construction of a shuttle plasmid pVax-ΔE3(L+R) with the E3 gene knocked out

A left arm (L-ΔE3) and a right arm (R-ΔE3) of the E3 gene were obtained by PCR amplification using an Ad14 genome as a template.

L-ΔE3 primer:

L-ΔE3-F,

(SEQ ID NO: 5)

GATATCTAGAGTGAATTCGTCCAAATGACTAATG
CAGGTGC;
L-ΔE3-R,

(SEQ ID NO: 6)

CCAGTAGAAGCGCCGGATTTAAATAGGAAAAGT
TTCGTTCTTCTGGTTG.

PCR conditions: 95° C., 3 min; 95° C., 30 s; 61° C., 30 s; 72° C., 50 s; cycles 30; 72° C., 5 min; stored at 12° C.

R-ΔE3 primer:

R-ΔE3-F,

(SEQ ID NO: 7)

ATTATTGACTAGAGTAATTTAAATGGACTAAGAG
ACCTGCTACCCATG;
R-ΔE3-R,

(SEQ ID NO: 8)

GAATTCACTCTAGATATCCCACTTTTAGCTGTAG
AGAC CCG.

PCR conditions: 95° C., 3 min; 95° C., 30 s; 60° C., 30 s; 72° C., 30 s; cycles 30; 72° C., 5 min; stored at 12° C.

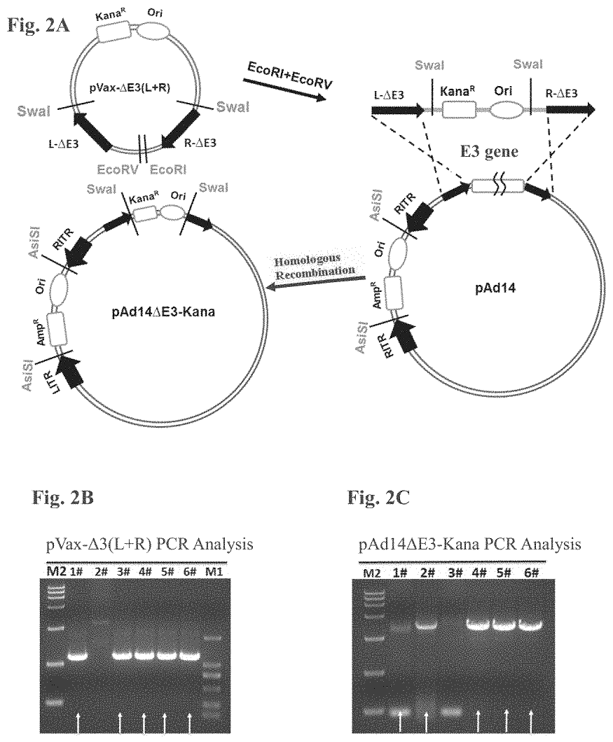

The L-ΔE3, R-ΔE3, and a plasmid skeleton obtained by digesting a pVax vector with Bstz17I +SgrAI through double enzyme digestion were subjected to a triple-fragment ligation using an Exnase enzyme to obtain pVax-ΔE3(L+R). Detection of pVax-ΔE3(L+R) by PCR assay were shown in FIG. 2B.

2. Construction of pAd14ΔE3-Kana

The pVax-ΔE3(L+R) was digested with EcoRI +EcoRV through double enzyme digestion, and the pAd14 was enzyme digested with EcoRI. Fragments recovered from the two enzyme digested products were co-transformed into a BJ5183 competent cell for recombination. The recombined BJ5183 competent cell was subjected to resistance screening by ampicillin and kanamycin dual-resistance plates. After amplification of the screened monoclone, the plasmid was extracted from the screened monoclone and transformed into an XL competent cell, from which a plasmid was extracted, to obtain pAd14ΔE3-Kana. The pAd14ΔE3-Kana was identified by different ways of enzyme digestion, and the pAd14ΔE3-Kana introduced two SwaI enzyme digestion sites at both sides of the kanamycin gene introduced in the E3 region, so as to facilitate subsequent cloning. See a schematic of plasmid construction in FIG. 2A and PCR analysis result of pAd14ΔE3-Kana in FIG. 2C.

### EXAMPLE 3

Construction of Plasmid pAd14ΔE3 with a Kanamycin Resistant Gene Knocked Out from pAd14ΔE3-Kana Plasmid

1. Construction of a shuttle plasmid pVax-ΔK(E3) with a kanamycin resistant gene knocked out from the E3 region

A left arm L-ΔK(E3) and a right arm R-ΔK(E3) of the E3 gene were obtained by PCR amplification using an Ad14 genome as a template.

L-ΔK(E3) primer:

L-ΔK(E3)-F,

(SEQ ID NO: 9)

TGATTATTGACTAGAGTATACGTCCAAATGACT
AATGCAGGTGC;
L-ΔK(E3)-R,

(SEQ ID NO: 10)

GATATCATTTAAATACTAGTAGGAAAAGTTTCGTTCT TCTGGTTG.

PCR conditions: 95° C., 3 min; 95° C., 30 s; 60° C., 30 s; 72° C., 50 s; cycles 30; 72° C., 5 min; stored at 12° C.

R-ΔK(E3) primer:

R-ΔK(E3)-F,

(SEQ ID NO: 11)

ACTAGTATTTAAATGATATCGGACTAAGAGAC
CTGCTACCCATG;
R-ΔK(E3)-R,

(SEQ ID NO: 12)

ACCGCCCAGTAGAAGCGCCGGTGCCACTTTT
AGCTGTAGAGACCCG.

PCR conditions: 95° C., 3 min; 95° C., 30 s; 60° C., 30 s; 72° C., 30 s; cycles 30; 72° C., 5 min; stored at 12° C.

The L-ΔK(E3), R-ΔK(E3), and a plasmid skeleton obtained by digesting a pVax vector with Bstz17I +SgrAI through double enzyme digestion were subjected to a triple-fragment ligation using an Exnase enzyme to obtain pVax-ΔK(E3).

2. Construction of pAd14ΔE3

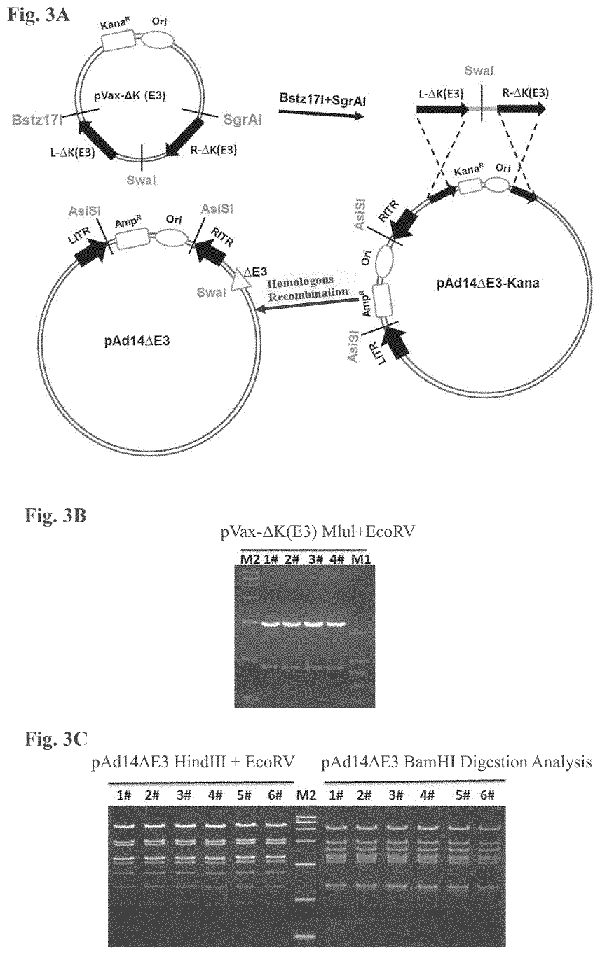

The pVax-ΔK(E3) was linearized with Bstz 17I +SgrAI through double enzyme digestion, and the pAd14ΔE3-Kana was linearized with SwaI through enzyme digestion. Fragments recovered from the two enzyme digested products were co-transformed into a BJ5183 competent cell for recombination. The recombined BJ5183 competent cell was subjected to resistance screening by an ampicillin resistant plate. After amplification of the screened monoclone, a plasmid was extracted from the screened monoclone and transformed into an XL competent cell, from which a plasmid was extracted, to obtain pAd14ΔE3. A plasmid was extracted from the pAd14ΔE3 and identified by enzyme digestion. The pAd14ΔE3 and pVax-ΔK(E3) were recombined with a single enzyme digestion site SwaI introduced in the E3 region while knocking out the kanamycin resistant gene. See a schematic of plasmid construction in FIG. 3A, endonuclease digestion result of pVax-ΔK(E3) with Mlul and EcoRV in FIG. 3B, and endonuclease digestion results of pAd14ΔE3 with HindIII and EcoRV and pAd14ΔE3 with BamH I in FIG. 3C.

### EXAMPLE 4

### Knocking Out of E1 Gene and Construction of pAd14ΔE1ΔE3-Kana Plasmid

1. Construction of a shuttle plasmid pVax-ΔE1(L+R) with the E1 gene knocked out

A left arm (L-ΔE1) and a right arm (R-ΔE1) of the E1 gene were obtained by PCR amplification using an Ad14 genome as a template.

L-ΔE1 primer:

L-ΔE1-F,

(SEQ ID NO: 13)

GATATCTAGAGTGAATTCGGGGTGGAGTGTTTTT
GCAAG;
L-ΔE1-R,

(SEQ ID NO: 14)

CGCCCAGTAGAAGCGCCGGGTTTAAACGTAATC
GAAACCTCCACGTAATGG.

PCR conditions: 95° C., 3 min; 95° C., 30 s; 62° C., 30 s; 72° C., 30 s; cycles 30; 72° C., 5 min; stored at 12° C.

R-ΔE1 primer:

R-ΔE1-F,

(SEQ ID NO: 15)

CCAGATATACGCGTGTATAGTTTAAACGAGACCG
GATCATTTGGTTATTG;
R-ΔE1-R,

(SEQ ID NO: 16)

GAATTCACTCTAGATATCGGGAAATGCAAATCTG
TGAG GG.

PCR conditions: 95° C., 3 min; 95° C., 30 s; 61° C., 30 s; 72° C., 1 min; cycles 30; 72° C., 5 min; stored at 12° C.

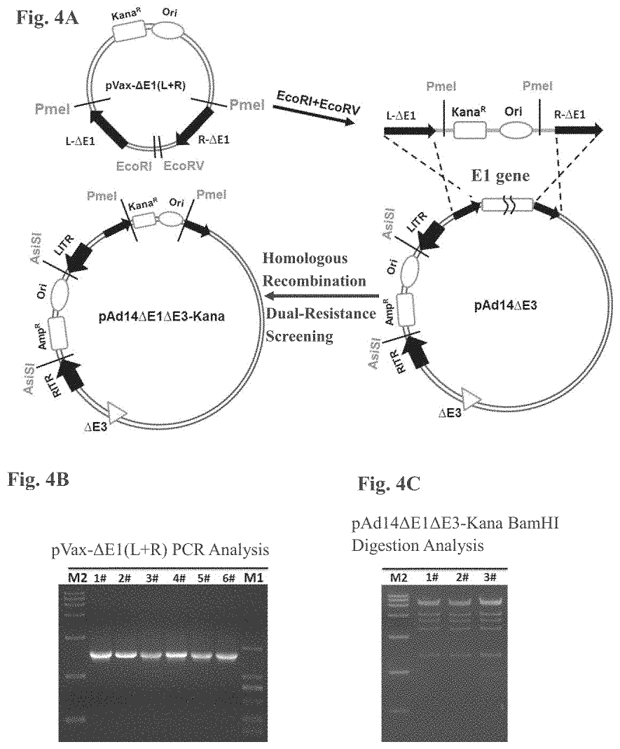

The L-ΔE1, R-ΔE1, and a plasmid skeleton obtained by digesting a pVax vector with Bstz17I +SgrAI through double enzyme digestion were subjected to a triple-fragment ligation using an Exnase enzyme to obtain pVax-ΔE1(L+R).Detection of pVax-ΔE1(L+R) by PCR assay were shown in FIG. 4B.

2. Construction of pAd14ΔE1ΔE3-Kana

The pVax-ΔE1(L+R) was linearized with EcoRI +EcoRV through double enzyme digestion, and the pAd14ΔE3 was linearized with PacI through enzyme digestion. Fragments recovered from the two enzyme digested products were co-transformed into a BJ5183 competent cell for recombination. The recombined BJ5183 competent cell was subjected to resistance screening by ampicillin and kanamycin dual-resistance plates. After amplification of the screened monoclone, a plasmid was extracted from the screened monoclone and transformed into an XL competent cell, from which a plasmid was extracted, to obtain pAd14ΔE1ΔE3-Kana. The pAd14ΔE1ΔE3-Kana was identified by different ways of enzyme digestion, and introduced two PmeI enzyme digestion sites at both sides of the kanamycin gene introduced in the E1 gene region, so as to facilitate subsequent cloning. See a schematic of plasmid construction in FIG. 4A and endonuclease digestion result of pAd14ΔE1ΔE3-Kana with BamH I in FIG. 4C.

### EXAMPLE 5

### Construction of Plasmid pAd14ΔE1ΔE3 with Kanamycin Resistant Gene Knocked Out from pAd14ΔE1ΔE3-Kana

1. Construction of a shuttle plasmid pVax-ΔK(E1) with a kanamycin resistant gene knocked out from the E1 region

A left arm L-ΔK(E1) and a right arm R-ΔK(E1) of the E1 gene were obtained by PCR amplification using an Ad14 genome as a template.

L-ΔK(E1) primer:

L-ΔK(E1)-F,

(SEQ ID NO: 17)

ACCGCCCAGTAGAAGCGCCGGTGGGGGTGG
AGTGTTTTTGCAAG;
L-ΔK(E1)-R,

(SEQ ID NO: 18)

ACTAGTGTTTAAACGATATCGTAATCGAAACC
TCCA CGTAATGG.

PCR conditions: 95° C., 3 min; 95° C., 30 s; 61° C., 30 s; 72° C., 30 s; cycles 30; 72° C., 5 min; stored at 12° C.

R-ΔK(E1) primer sequence:

R-ΔK(E1)-F,

(SEQ ID NO: 19)

GATATCGTTTAAACACTAGTGAGACCGGATCA
TTTGGTTATTG;
R-ΔK(E1)-R,

(SEQ ID NO: 20)

CCAGATATACGCGTGTATACGGGAAATGCAAA
TCTGTGAGGG.

PCR conditions: 95° C., 3 min; 95° C., 30 s; 61° C., 30 s; 72° C., 1 min; cycles 30; 72° C., 5 min; stored at 12° C.

The L-ΔK(E1), R-ΔK(E1), and a plasmid skeleton obtained by digesting a pVax vector with Bstz17I +SgrAI through double enzyme digestion were subjected to a triple-fragment ligation using an Exnase enzyme to obtain pVax-ΔK(E1).

2. Construction of pAd14ΔE1ΔE3

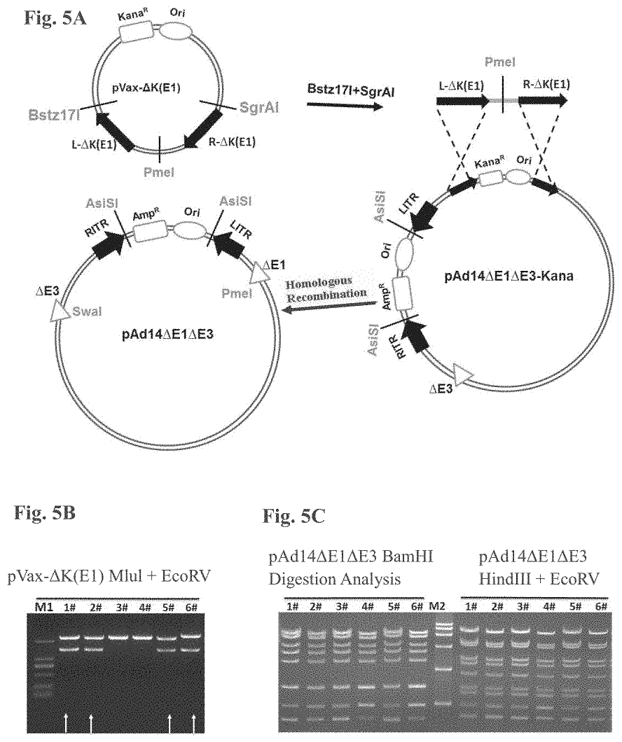

The pVax-ΔK(E1) was linearized with Bstz 17I +SgrAI through double enzyme digestion, and the pAd14ΔE1ΔE3-Kana was linearized with PmeI through enzyme digestion. Fragments recovered from the two enzyme digested products were co-transformed into a BJ5183 competent cell for recombination. The recombined BJ5183 competent cell was subjected to resistance screening by an ampicillin resistant plate. After amplification of the screened monoclone, the plasmid was extracted from the screened monoclone and transformed into an XL competent cell, from which a plasmid was extracted, to obtain pAd14ΔE1ΔE3. A plasmid was extracted from the pAd14ΔE1ΔE3 and identified by enzyme digestion. The pAd14ΔE1ΔE3 and pVax-ΔK(E1) were recombined with a single enzyme digestion site SwaI introduced in the E1 region while knocking out the kanamycin resistant gene. See a schematic of plasmid construction in FIG. 5A, endonuclease digestion result of pVax-ΔK(E1) with Mlul and EcoRV in FIG. 5B, and endonuclease digestion results of pAd14ΔE1ΔE3 with BamH I and pAd14ΔE1ΔE3 with HindIII and EcoRV in FIG. 5C.

### EXAMPLE 6

### Modification of Ad14 E4 Gene and Construction of pAd14ΔE1ΔE3(5E4)

1. Construction of a shuttle plasmid p14E4(5ORF2-6) integrated with Ad5 E4 ORF2-6

1) An E4 gene of Ad14 was obtained by PCR amplification using an Ad14 genome as a template.

Ad14 E4 primer:

E4-F,

(SEQ ID NO: 21)

AGAGTGCACCATATGGAATTCGGACTAAGAGACCTG
CTACCCATG;
E4-R,

(SEQ ID NO: 22)

GCTGTGTGGTAATTGGCTGTGGG.

PCR conditions: 95° C., 3 min; 95° C., 30 s; 62° C., 30 s; 72° C., 4 min; cycles 30; 72° C., 5 min; stored at 12° C.

An end of the E4 gene fragment obtained by PCR amplification was phosphorized, and the phosphorized end was ligated into a T vector at the flat end to obtain p14E4.

2) By using the following primers, Ad5 E4 ORF2-6 was obtained by PCR amplification using an Ad5 genome as a template.

5(ORF2-6)-F,

(SEQ ID NO: 23)

CATTCAAAACTAACAATGCAGAAACCCGCAG
ACATG;
5(ORF2-6)-R,

(SEQ ID NO: 24)

GAGTCTGGATCACGGCTACATGGGGGTAGA
GTCATAATCG.

PCR conditions: 95° C., 3 min; 95° C., 30 s; 62° C., 30 s; 72° C., 2 min; cycles 30; 72° C., 5 min; stored at 12° C.

3) By using the following primers, a p14E4 skeleton was obtained by PCR amplification using the p14E4 as a template.

p14E4-F,

(SEQ ID NO: 25)

CCGTGATCCAGACTCCGGAG;
p14E4-R,

(SEQ ID NO: 26)

TGTTAGTTTTGAATGAGTCTGCAAA.

PCR conditions: 95° C., 3 min; 95° C., 30 s; 60° C., 30 s; 72° C., 5 min; cycles 30; 72° C., 5 min; stored at 12° C.

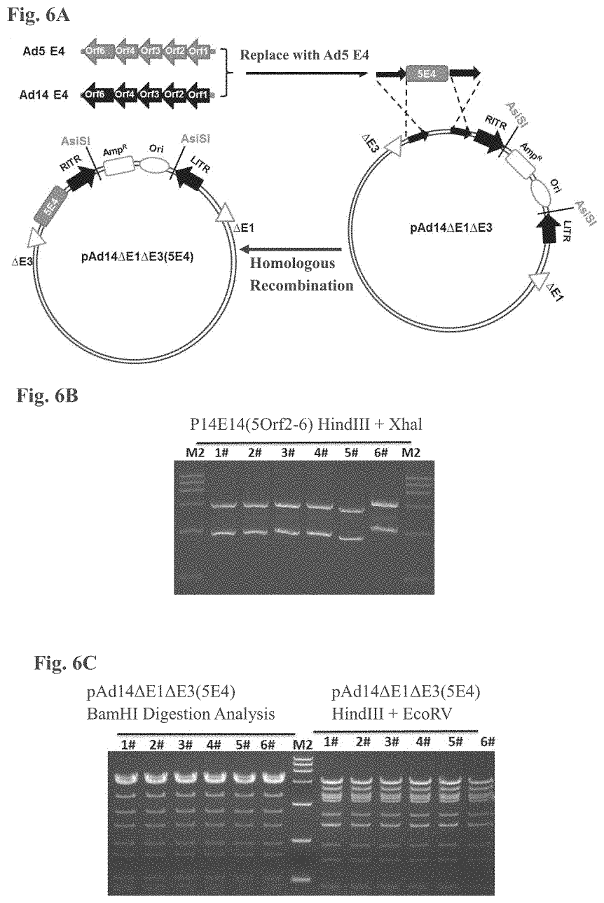

The 5(ORF2-6) and the p14E4 plasmid skeleton were subjected to a double ligation with Exnase to obtain p14E4(5Orf2-6). The endonuclease digestion result of p14E4(5Orf2-6) by HindIII and XbaI was shown in FIG. 6B.

2. Construction of a plasmid pAd14ΔE1ΔE3(5E4)

The p14E4(5Orf2-6) was linearized with EcoRI, and the pAd14ΔE1ΔE3 was linearized with PsiI. Fragments recovered from the two enzyme digested products were co-transformed into a BJ5183 competent cell for recombination. The recombined BJ5183 competent cell was subjected to resistance screening by an ampicillin resistant plate. After amplification of the screened monoclone, the plasmid was extracted from the screened monoclone and transformed into an XL competent cell, from which a plasmid was extracted, to obtain pAd14ΔE1ΔE3(5E4). A plasmid was extracted from the pAd14ΔE1ΔE3(5E4) and identified by enzyme digestion. See a schematic of plasmid construction in FIG. 6A and endonuclease digestion results of pAd14ΔE1ΔE3(5E4) with BamH I and pAd14ΔE1ΔE3(5E4) with HindIII and EcoRV in FIG. 6C.

### EXAMPLE 7

### Construction of shuttle plasmid harboring exogenous genes such as EGFP and construction of replication defective Ad14 genomic plasmid

pAd14ΔE14E3(5E4)-EGFP

1. Construction of a shuttle plasmid pSE1LR harboring recombination arms at both sides of the E1 gene

A left arm (L-SE1) and a right arm (R-SE1) of the E1 gene were obtained by PCR amplification using an Ad14 genome as a template.

L-SE1 primer:

L-SE1-F,

(SEQ ID NO: 27)

ACCGCCCAGTAGAAGCGCCGGTGGGGGTGGAGT
GTTTTTGCAAG;
L-SE1-R,

(SEQ ID NO: 28)

ACTAGTGTTTAAACGATATCGTAATCGAAACCTC
CACGTAATGG.

PCR conditions: 95° C., 3 min; 95° C., 30 s; 61° C., 30 s; 72° C., 30 s; cycles 30; 72° C., 5 min; stored at 12° C.

R-SE1 primer:

R-SE1-F,

(SEQ ID NO: 29)

GATATCGTTTAAACACTAGTGAGACCGGATCATT
TGGTTATTG;
R-SE1-R,

(SEQ ID NO: 30)

CCAGATATACGCGTGTATACGGGAAATGCAAATC
TGTGAGGG.

PCR conditions: 95° C., 3 min; 95° C., 30 s; 61° C., 30 s; 72° C., 1 min; cycles 30; 72° C., 5 min; stored at 12° C.

The L-SE1, R-SE1, and a plasmid skeleton obtained by digesting a pVax vector with Bstz17I +SgrAI through double enzyme digestion were subjected to a triple-fragment ligation with an Exnase enzyme to obtain pSE1(L+R).

2. Construction of a shuttle plasmid pGK141-EGFP harboring an exogenous gene such as EGFP

The CMV-EGFP-BGH was obtained by PCR amplification using pGA1-EGFP as a template.

Primer sequence:

CMV-EGFP-BGH-F,

(SEQ ID NO: 31)

AGATATACGCGTTGACATTGATTATTGA
CTAG;
CMV-EGFP-BGH-R,

(SEQ ID NO: 32)

GCTGGTTCTTTCCGCCTCAGAAG.

PCR conditions: 95° C., 3 min; 95° C., 30 s; 65° C., 30 s; 72° C., 1 min 50 s; cycles 30; 72° C., 5 min; stored at 12° C.

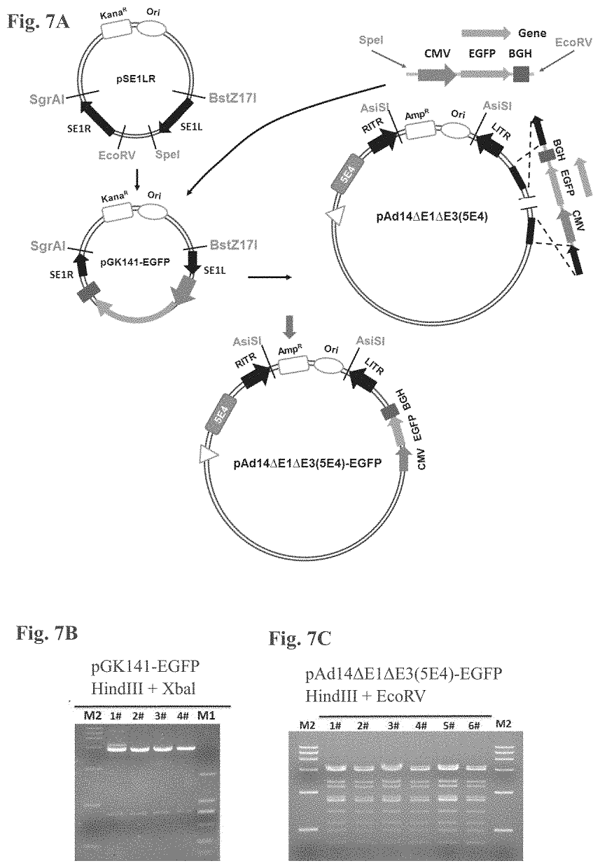

The CMV-EGFP-BGH expression cassette was digested with SpeI through enzyme digestion, and the pSE1(L+R) was digested with HindIII+XbaI through double enzyme digestion. The two enzyme digested products were ligated to obtain pGK141-EGFP. The endonuclease digestion result of pGK141-EGFP by HindIII and XbaI was shown in FIG. 7B.

3.Construction of pAd14ΔE1ΔE3(5E4)-EGFP

The pGK141-EGFP was linearized by Bstz17I +SgrAI, and the pAd14ΔE1ΔE3(5E4) was linearized by PmeI. Fragments recovered from the two enzyme digested products were co-transformed into a BJ5183 competent cell for recombination. The recombined BJ5183 competent cell was subjected to resistance screening by an ampicillin resistant plate. After amplification of the screened monoclone, the plasmid was extracted from the screened monoclone and transformed into an XL competent cell, from which a plasmid was extracted, to obtain pAd14ΔE1ΔE3(5E4)-EGFP. A plasmid was extracted from the pAd14ΔE1ΔE3(5E4)-EGFP and identified by enzyme digestion. See a schematic of plasmid construction in FIG. 7A and endonuclease digestion result of pAd14ΔE1ΔE3(5E4)-EGFP with HindIII and EcoRV in FIG. 7C.

### EXAMPLE 8

### Rescue and Production of Replication Defective Ad14 Vectors

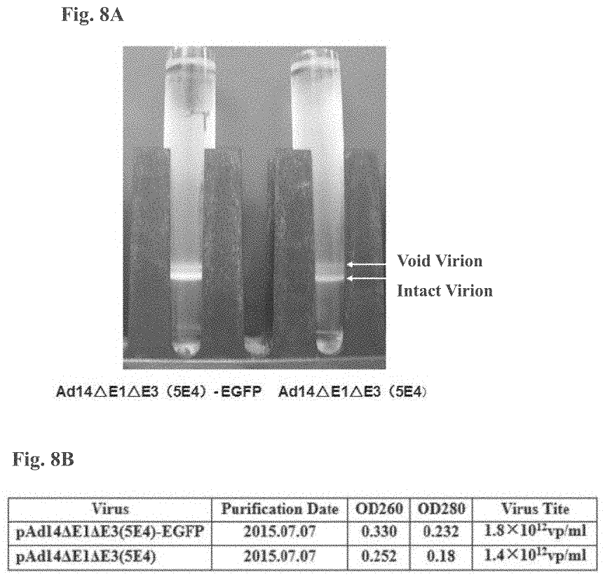

The pAd14ΔE1ΔE3(5E4) and pAd14ΔE1ΔE3(5E4)-EGFP were linearized with AsisI through enzyme digestion, respectively, then recovered by ethanol precipitation, and transfected into 293 cells by using Lipofectamine2000. The 293 cells were cultured in a DMEM medium containing 5% FBS 8h later of transfection. From the 7^{th} day of culture, the cells were observed every day to look for cytopathic effect (CPE). Once obviously CPE were observed, the cells and culture supernatant were collected, and repeatedly frozen and thawed for 3 times in a liquid nitrogen container and a water bath kettle at 37° C., and then cell debris was removed by centrifugation. A suitable amount of supernatant was added to a 10 cm cell-culture dish to infect 293 cells. When the CPE was observed after infection for 2 to 3 days, the cells and supernatant were collected and repeatedly frozen and thawed for 3 times, and thereafter, cell debris was removed by centrifugation. A supernatant was collected and added to 15 cm cell-culture dishes to infect eight to nine 293 cells for propagation. When the CPE was observed after infection for 2 to 3 days, the cells and supernatant were collected and repeatedly frozen and thawed for 3 times, and thereafter, cell debris was removed by centrifugation. A supernatant was collected and added in a centrifuge tube of cesium chloride gradient, balanced, and centrifuged for 4 hours at 4° C., 30000 rpm. After centrifugation, a virus band was extracted carefully, desalted and packaged. An appropriate amount of the virus was used for OD260 concentration measurement, and virus concentration was calculated by the equation below: virus concentration =OD260×dilution factor×36/genome length (Kb). The collected and purified virus was stored at −80° C. The production result of a type 14 replication defective vector was shown in FIG. 8A. The purification result of a type 14 replication defective vector was shown in FIG. 8B.

### EXAMPLE 9

### Replication Assay of Replication Defective Ad14 in 293 and A549 Cells

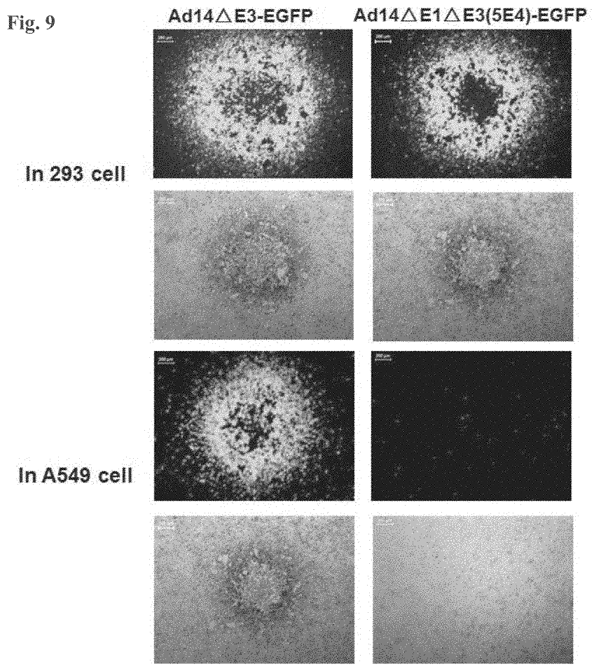

Replication capacities of replication defective Ad14 vectors in helper cell 293 and non-helper cell A549 were detected by a plaque assay. 293 or A549 cells were cultured in a 12-well cell plate. When the cell density approximated to 100%, the harvested P1-generation Ad14ΔE1ΔE3(5E4)-EGFP virus stock solution was undergone gradient dilution, and then infected into 293 or A549 cells, respectively. Duplicated wells were performed for each virus concentration. After the virus was infected into cells for 2h, the medium was removed, and each well was covered with 1.2% agarose gel about 1 ml (containing 1.2% agarose, 5% fetal bovine serum, 1×MEM medium, 1×penicillin-streptomycin). After about 9 to 12 days of culture, a green fluorescence expression in the virus was observed and formation of virus clone was sought by fluorescence microscope. Graphs were taken. The results of virus plaques formed by type 14 replication defective vector in 293 and A549 cells were shown in FIG. 9. The Ad14ΔE3-EGFP with only E3 gene knocked out could form typical virus plaques in both 293 and A549 cells, and thus had a replication capacity; the Ad14ΔE1ΔE3(5E4)-EGFP with both E1 and E3 genes knocked out could form virus plaques only in helper cells 293, but not in normal human A549 cells. The above results indicate that a replication defective Ad14 vector can replicate in a helper cell 293, but it cannot replicate in normal human cells such as A549 cells, and thus it exhibits an attenuated phenotype. In addition, a replication defective Ad14 vector can express the carried reporter genes in the infected cells and thus can be applied in biological report and trace systems.

### EXAMPLE 10

### Immunogenicity Evaluation of Replication Defective Ad14 Vectors in Mice

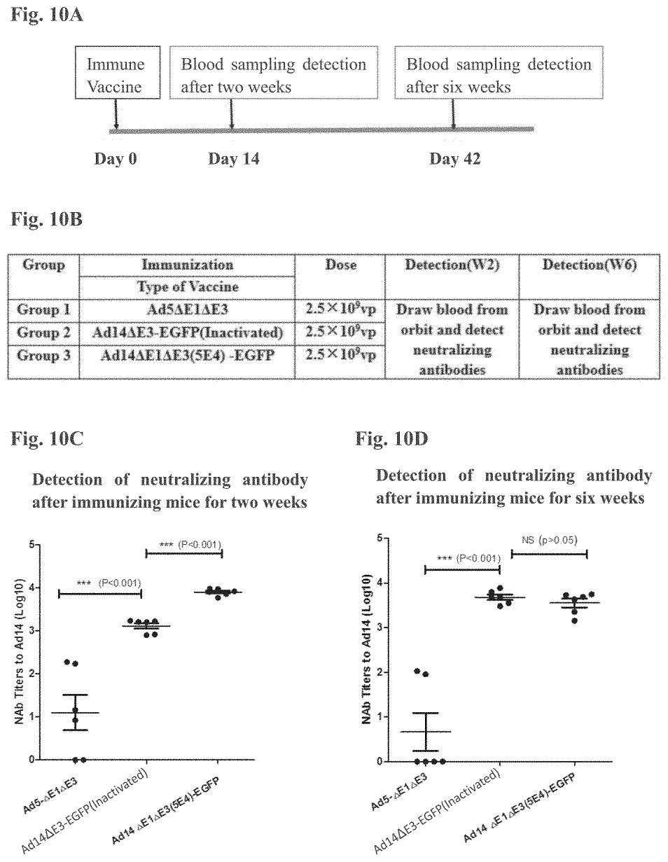

A scheme of evaluating immunogenicity of replication defective Ad14 in mice was designed (shown in the FIG. 10A and FIG. 10B). The immunogenicity of the replication defective Ad14 was evaluated according to the designed immunization scheme.

Balb/c mice at 6-8 weeks of age was selected and divided into 3 groups and each group included 6 mice. By intramuscular injection, mice in Group 1 was immunized with Ad5ΔE1ΔE3; mice in Group 2 was immunized with heat-inactivated Ad14ΔE3-EGFP; and mice in Group 3 was immunized with Ad14ΔE1ΔE3(5E4)-EGFP. On day 14 after immunization, blood samples were collected from orbit and the serum was separated, and neutralizing antibody against Ad14 was measured. Compared with the inactivated Ad14ΔE3-EGFP, Ad14ΔE1ΔE3(5E4)-EGFP could induce higher-level neutralizing antibodies against Ad14. On day 42 after immunization, blood samples were collected again from orbit, and valence of the neutralizing antibodies against Ad14 in serum was measured. The detection result of neutralizing antibody titer after immunizing mice for two weeks by replication defective Ad14ΔE1ΔE3(5E4) was shown in FIG. 10C. The detection result of neutralizing antibody titer after immunizing mice for six weeks by replication defective Ad14ΔE1ΔE3(5E4) was shown in FIG. 10D. At this moment, Ad14ΔE1ΔE3(5E4)-EGFP and inactivated Ad14ΔE3-EGFP could induce equivalent level of valence of neutralizing antibodies against Ad14. These results indicate that compared with the inactivated Ad14ΔE3-EGFP, Ad14ΔE1ΔE3(5E4)-EGFP can induce higher-level neutralizing antibodies against Ad14 at the early stage of immunizing mice.

The examples described above are merely illustrations of several embodiments of the present invention, and the specific and detailed examples are not intended to limit the scope of the patent invention. It should be noted that within the scope of the present patent invention, a number of modifications and variations may occur to one skilled in the art without departing from the scope and inventive ideas of the present patent invention. Accordingly, the scope of the present patent invention should be subject to the appended claims.

1. A human type 14 replication defective adenovirus vector, prepared by the following method: constructing an Ad14 genome into a plasmid, with knocking out E3 and E1 genes of the Ad14 genome, and replacing open reading frames 2, 3, 4,6, and 6/7 of an E4 gene of the Ad14 genome with corresponding reading frames of an Ad5 genome.

2. The human type 14 replication defective adenovirus vector according to claim 1, wherein the method further comprises integrating an exogenous gene expression cassette into an E1 gene region of Ad14.

3. A method of preparing the human type 14 replication defective adenovirus vector according to claim 1, comprising the following steps:
S1. obtaining left and right ends of the Ad14 genome by PCR amplification, ligating the ends into an ampicillin resistant plasmid to obtain pT-Ad14(L+R), linearizing the pT-Ad14(L+R), and recombining the linearized pT-Ad14(L+R) with the Ad14 genome to obtain a genomic plasmid pAd14;
S2. obtaining left and right arms of an Ad14 E3 gene by PCR amplification, ligating the arms in a reverse direction into a kanamycin resistant plasmid, linearizing the plasmid, and obtaining a genomic plasmid pAd14ΔE3-Kana with the E3 gene of Ad14 knocked out, through homologous recombination of the linearized plasmid with pAd14 which is linearized by partial enzyme digestion;
S3. obtaining left and right arms of an Ad14 E3 gene by PCR amplification, ligating the arms in a forward direction into the kanamycin resistant plasmid, linearizing the plasmid, and obtaining a genomic plasmid pAd14ΔE3 with a kanamycin resistant gene knocked out, through recombination of the linearized plasmid with the linearized pAd14ΔE3-Kana;
S4. obtaining left and right arms of an Ad14 E1 gene by PCR amplification, ligating the arms in a reverse direction into a kanamycin resistant plasmid, linearizing the plasmid, and obtaining a genomic plasmid pAd14ΔE1ΔE3-Kana with the E1 gene of Ad14 knocked out, through homologous recombination of the linearized plasmid with pAd14ΔE3 which is linearized by partial enzyme digestion;
S5. obtaining left and right arms of an Ad14 E1 gene by PCR amplification, ligating the arms in a forward direction into a kanamycin resistant plasmid, linearizing the plasmid, and obtaining a genomic plasmid pAd14ΔE1ΔE3 with a kanamycin resistant gene knocked out, through recombination of the linearized plasmid with the linearized pAd14ΔE1ΔE3-Kana; and
S6. obtaining an Ad14 E4 gene by PCR amplification and ligating the Ad14 E4 gene into an ampicillin resistant plasmid to obtain p14E4; obtaining open reading frames 2, 3, 4,6, and 6/7 of E4 gene of an Ad5 genome by PCR amplification, replacing corresponding regions of the Ad14 E4 gene to obtain p14E4(5E4), linearizing the p14E4(5E4), and obtaining a genomic plasmid pAd14ΔE1ΔE3(5E4) with E1 and E3 genes knocked out and E4 gene replaced, through homologous recombination of the linearized p14E4(5E4) with the linearized pAd 14ΔE1ΔE3.

4. The preparation method according to claim 3, wherein said step S1 comprises: obtaining left and right ends L-Ad14 and R-Ad14 as recombination arms of the Ad14 genome by PCR amplification using the Ad14 genome as a template, ligating the arms into a linearized T vector to obtain pT-Ad14(L+R), while introducing EcoRI and BamHI sites as enzyme digestion sites between left and right arms of the pT-Ad14(L+R), digesting the pT-Ad14(L+R) with EcoRI+BamHI through double enzyme digestion, and recombining with the Ad14 genome after linearization of the double enzyme digestion to obtain pAd14.

5. The preparation method according to claim 3, wherein said step S2 comprises: obtaining homologous recombination arms L-ΔE3 and R-ΔE3of the E3 gene by PCR amplification using the Ad14 genome as a template, ligating the arms in a reverse direction into a pVax vector to obtain pVax-ΔE3(L+R), linearizing the pVax-ΔE3(L+R), and obtaining a plasmid pAd14ΔE3-Kana with the E3 gene knocked out and a unique linearized enzyme digestion site SwaI introduced in the E3 gene region, through homologous recombination of the linearized pVax-ΔE3(L+R) with pAd14 linearized by partial enzyme digestion using EcoRI and a dual-resistance screening by ampicillin and kanamycin.

6. The preparation method according to claim 3, wherein said step S3 comprises: obtaining homologous recombination arms L-ΔK(E3) and R-ΔK(E3) of the E3 gene by PCR amplification using the Ad14 genome as a template, ligating the arms in a forward direction into a pVax vector to obtain pVax-ΔK(E3), linearizing the pVax-ΔK(E3), and obtaining pAd14ΔE3 with E3 and kanamycin resistant genes knocked out and a single enzyme digestion site SwaI introduced, through recombination of the linearized pVax-ΔK(E3) with pAd14ΔE3-Kana linearized by SwaI; and wherein said step S4 comprises: according to the same principle as in step S2, obtaining homologous recombination arms L-ΔE1 and R-ΔE1 of the E1 gene by PCR amplification, ligating in reverse direction into a pVax vector to obtain pVax-ΔE1(L+R), linearizing the pVax-ΔE1(L+R), and obtaining a plasmid pAd14ΔE1ΔE3-Kana with the E1 gene knocked out and a unique linearized enzyme digestion site PmeI introduced in the E1 gene region, through homologous recombination of the linearized pVax-ΔE1(L+R) with pAd14ΔE3 linearized by enzyme digestion using PacI and a dual-resistance screening by ampicillin and kanamycin.

7. The preparation method according to claim 3, wherein said step S5 comprises: according to the same principle as in step S3, obtaining homologous recombination arms L-ΔK(E1) and R-ΔK(E1) of the E1 gene by PCR amplification, ligating the arms in a forward direction into a pVax vector to obtain pVax-ΔK(E1), linearizing the pVax-ΔK(E1), and obtaining pAd14ΔE1ΔE3 with E1 and kanamycin resistant genes knocked out and a single enzyme digestion site PmeI introduced, through recombination of the linearized pVax-ΔK(E1) with pAd14ΔE1ΔE3-Kana linearized by PmeI; and wherein said step S6 comprises: obtaining Ad5 E4 Orf2-6 and Ad14 E4 by PCR amplification using Ad5 genome and Ad14 genome as templates respectively, ligating the Ad14 E4 into a T vector to obtain p14E4, further knocking out Ad14 E4 Orf2-6 by PCR using p14E4 as a template, then ligating the PCR product with Ad5 E4 Orf2-6 to obtain p14E4(5E4), linearizing the p14E4(5E4), and obtaining pAd14ΔE1ΔE3(5E4), through homologous recombination of the linearized p14E4(5E4) with the linearized pAd14ΔE1ΔE3.

8. The preparation method according to claim 3, wherein the method further comprises: integrating into an exogenous sequence through homologous recombination, further comprising the following step:
S7. obtaining homologous recombination arms L-SE1 and R-SE1 of the E1 region by PCR using the Ad14 genome as a template, enzyme digesting the arms and ligating the enzyme digested arms into a pVax vector to obtain pSE1LR; producing an exogenous gene expression cassette CMV-EGFP-BGH by PCR using pGA1-EGFP as a template, enzyme digesting the CMV-EGFP-BGH and pSE1LR, ligating the digested CMV-EGFP-BGH and pSE1LR to obtain pGK141-EGFP, linearizing the pGK141-EGFP, and obtaining pAd14ΔE1ΔE3(5E4)-EGFP through homologous recombination of the linearized pGK14I-EGFP with the linearized pAd14ΔE1ΔE3(5E4)obtained in step S6, and then transfecting a cell after further linearization, culturing the transfected cell and obtaining Ad14ΔE1ΔE3(5E4)-EGFP by centrifugal purification.

9. A method of preparing the human type 14 replication defective adenovirus vector according to claim 2, comprising the following steps:
S1. obtaining left and right ends of the Ad14 genome by PCR amplification, ligating the ends into an ampicillin resistant plasmid to obtain pT-Ad14(L+R), linearizing the pT-Ad14(L+R), and recombining the linearized pT-Ad14(L+R) with the Ad14 genome to obtain a genomic plasmid pAd14;
S2. obtaining left and right arms of an Ad14 E3 gene by PCR amplification, ligating the arms in a reverse direction into a kanamycin resistant plasmid, linearizing the plasmid, and obtaining a genomic plasmid pAd14ΔE3-Kana with the E3 gene of Ad14 knocked out, through homologous recombination of the linearized plasmid with pAd14 which is linearized by partial enzyme digestion;
S3. obtaining left and right arms of an Ad14 E3 gene by PCR amplification, ligating the arms in a forward direction into the kanamycin resistant plasmid, linearizing the plasmid, and obtaining a genomic plasmid pAd14ΔE3 with a kanamycin resistant gene knocked out, through recombination of the linearized plasmid with the linearized pAd14ΔE3-Kana;
S4. obtaining left and right arms of an Ad14 E1 gene by PCR amplification, ligating the arms in a reverse direction into a kanamycin resistant plasmid, linearizing the plasmid, and obtaining a genomic plasmid pAd14ΔE1ΔE3-Kana with the E1 gene of Ad14 knocked out, through homologous recombination of the linearized plasmid with pAd14ΔE3 which is linearized by partial enzyme digestion;
S5. obtaining left and right arms of an Ad14 E1 gene by PCR amplification, ligating the arms in a forward direction into a kanamycin resistant plasmid, linearizing the plasmid, and obtaining a genomic plasmid pAd14ΔE1ΔE3 with a kanamycin resistant gene knocked out, through recombination of the linearized plasmid with the linearized pAd14ΔE1ΔE3-Kana; and
S6. obtaining an Ad14 E4 gene by PCR amplification and ligating the Ad14 E4 gene into an ampicillin resistant plasmid to obtain p14E4; obtaining open reading frames 2, 3, 4, 6, and 6/7 of E4 gene of an Ad5 genome by PCR amplification, replacing corresponding regions of the Ad14 E4 gene to obtain p14E4(5E4), linearizing the p14E4(5E4), and obtaining a genomic plasmid pAd14ΔE1ΔE3(5E4) with E1 and E3 genes knocked out and E4 gene replaced, through homologous recombination of the linearized p14E4(5E4) with the linearized pAd14ΔE1 ΔE3.

10. The preparation method according to claim 9, wherein said step S1 comprises: obtaining left and right ends L-Ad14 and R-Ad14 as recombination arms of the Ad14 genome by PCR amplification using the Ad14 genome as a template, ligating the arms into a linearized T vector to obtain pT-Ad14(L+R), while introducing EcoRI and BamHI sites as enzyme digestion sites between left and right arms of the pT-Ad14(L+R), digesting the pT-Ad14(L+R) with EcoRI+BamHI through double enzyme digestion, and recombining with the Ad14 genome after linearization of the double enzyme digestion to obtain pAd14.

11. The preparation method according to claim 9, wherein said step S2 comprises: obtaining homologous recombination arms L-ΔE3 and R-ΔE3of the E3 gene by PCR amplification using the Ad14 genome as a template, ligating the arms in a reverse direction into a pVax vector to obtain pVax-ΔE3(L+R), linearizing the pVax-ΔE3(L+R), and obtaining a plasmid pAd14ΔE3-Kana with the E3 gene knocked out and a unique linearized enzyme digestion site SwaI introduced in the E3 gene region, through homologous recombination of the linearized pVax-ΔE3(L+R) with pAd14 linearized by partial enzyme digestion using EcoRI and a dual-resistance screening by ampicillin and kanamycin.

12. The preparation method according to claim 9, wherein said step S3 comprises: obtaining homologous recombination arms L-ΔK(E3) and R-ΔK(E3) of the E3 gene by PCR amplification using the Ad14 genome as a template, ligating the arms in a forward direction into a pVax vector to obtain pVax-ΔK(E3), linearizing the pVax-ΔK(E3), and obtaining pAd14ΔE3 with E3 and kanamycin resistant genes knocked out and a single enzyme digestion site SwaI introduced, through recombination of the linearized pVax-ΔK(E3) with pAd14ΔE3-Kana linearized by SwaI; and wherein said step S4 comprises: according to the same principle as in step S2, obtaining homologous recombination arms L-ΔE1 and R-ΔE1 of the E1 gene by PCR amplification, ligating in reverse direction into a pVax vector to obtain pVax-ΔE1(L+R), linearizing the pVax-ΔE1(L+R), and obtaining a plasmid pAd14ΔE1ΔE3-Kana with the E1 gene knocked out and a unique linearized enzyme digestion site PmeI introduced in the E1 gene region, through homologous recombination of the linearized pVax-ΔE1(L+R) with pAd14ΔE3 linearized by enzyme digestion using PacI and a dual-resistance screening by ampicillin and kanamycin.

13. The preparation method according to claim 9, wherein said step S5 comprises: according to the same principle as in step S3, obtaining homologous recombination arms L-ΔK(E1) and R-ΔK(E1) of the E1 gene by PCR amplification, ligating the arms in a forward direction into a pVax vector to obtain pVax-ΔK(E1), linearizing the pVax-ΔK(E1), and obtaining pAd14ΔE1ΔE3 with E1 and kanamycin resistant genes knocked out and a single enzyme digestion site PmeI introduced, through recombination of the linearized pVax-ΔK(E1) with pAd14ΔE1ΔE3-Kana linearized by PmeI; and wherein said step S6 comprises: obtaining Ad5 E4 Orf2-6 and Ad14 E4 by PCR amplification using Ad5 genome and Ad14 genome as templates respectively, ligating the Ad14 E4 into a T vector to obtain p14E4, further knocking out Ad14 E4 Orf2-6 by PCR using p14E4 as a template, then ligating the PCR product with Ad5 E4 Orf2-6 to obtain p14E4(5E4), linearizing the p14E4(5E4), and obtaining pAd14ΔE1ΔE3(5E4), through homologous recombination of the linearized p14E4(5E4) with the linearized pAd14ΔE1ΔE3.

14. The preparation method according to claim 9, wherein the method further comprises: integrating into an exogenous sequence through homologous recombination, further comprising the following step:
S7. obtaining homologous recombination arms L-SE1 and R-SE1 of the E1 region by PCR using the Ad14 genome as a template, enzyme digesting the arms and ligating the enzyme digested arms into a pVax vector to obtain pSE1LR; producing an exogenous gene expression cassette CMV-EGFP-BGH by PCR using pGA1-EGFP as a template, enzyme digesting the CMV-EGFP-BGH and pSE1LR, ligating the digested CMV-EGFP-BGH and pSE1LR to obtain pGK141-EGFP, linearizing the pGK141-EGFP, and obtaining pAd14ΔE1ΔE3(5E4)-EGFP through homologous recombination of the linearized pGK141-EGFP with the linearized pAd14ΔE1ΔE3(5E4)obtained in step S6, and then transfecting a cell after further linearization, culturing the transfected cell and obtaining Ad14ΔE1ΔE3(5E4)-EGFP by centrifugal purification.

15. A vaccine, a neutralizing antibody, or a biological report and trace system prepare by using the human type 14 replication defective adenovirus vector according to claim 1.

16. A vaccine against human type 14 adenovirus or a drug against human type 14 adenovirus prepare by using the human type 14 replication defective adenovirus vector according to claim 1.

17. A vaccine, a neutralizing antibody, or a biological report and trace system prepare by using the human type 14 replication defective adenovirus vector according to claim 2.

18. A vaccine against human type 14 adenovirus or a drug against human type 14 adenovirus prepare by using the human type 14 replication defective adenovirus vector according to claim 2.
